(19) United States
(12) Patent Application Publication
(10) Pub. No.: US 2005/0080314 A1
(43) Pub. Date: Apr. 14, 2005

(54) Shielded transport for multiple brachytheapy implants with integrated measuring and cutting board

(76) Inventors: Richard A. Terwilliger, Southbury, CT (US); Gary A. Lamoureux, Woodbury, CT (US)

Correspondence Address: FLIESLER MEYER, LLP, FOUR EMBARCADERO CENTER, SUITE 400, SAN FRANCISCO, CA, 94111, US

(21) Appl. No.: 10/682,359

(22) Filed: Oct. 9, 2003

(51) Int. Cl. A61M 36/12; G21F 5/00

(52) U.S. Cl. 600/7; 250/506.1

(57) ABSTRACT

A normal system 100 for sterilizing, transporting and preparing radioactive seed strands for use in brachytherapy is provided. The system 100 is lightweight and can carry a plurality of seed strands and strand of spacer material, and can be sterilized by Ethylene Oxide Gas. Radioactive seed strands remain shielded from the user until the user is ready to retrieve each individual seed strand for measuring and cutting on an integrated cutting board.

### CROSS-REFERENCE TO RELATED APPLICATIONS

The following applications are cross-referenced and incorporated herein by reference as if fully set forth herein:

U.S. Patent Application No. 60/360,237, entitled “System for Manufacturing Interstitial Radiation Therapy Seed Strands”, by Terwilliger et al., filed Feb. 26, 2002.

U.S. Patent Application No. 60/360,272 entitled “Delivery System and Method for Interstitial Radiation Therapy Using Strands Constructed with Extruded Strand Housing”, by Terwilliger et al., filed Feb. 26, 2002.

U.S. Patent Application No. 60/360,241 entitled “Delivery System and Method for Interstitial Radiation Therapy Using Seed Strands Constructed with Preformed Strand Housing”, by Terwilliger et al., filed Feb. 26, 2002.

U.S. patent application Ser. No. 10/132,930 entitled “Improved Delivery System and Method for Interstitial Radiotherapy Using Hollow Seeds”, by Terwilliger et al., filed Apr. 26, 2003.

U.S. patent application Ser. No. 10/162,548 entitled “Delivery System and Method for Interstitial Radiation Therapy Using Seed Strands Constructed with Preformed Strand Housing”, by Terwilliger et al., filed Jun. 4, 2002.

U.S. patent application Ser. No. 10/162,546 entitled, “System for Manufacturing Interstitial Radiation Therapy Seen Strands” by Terwilliger et al., filed Jun. 4, 2002.

U.S. patent application Ser. No. 10/162,006 entitled, “Delivery System and Method for Interstitial Radiation Therapy Using Strands Constructed with Extruded Strand Housings”, by Terwilliger et al, filed Jun. 4, 2002.

### FIELD OF INVENTION

The present invention relates to systems and methods for delivering a plurality of radioactive sources to a treatment site, and to methods of, sterilizing, transporting and customizing such radioactive sources.

### BACKGROUND

In interstitial radiation therapy, one method for treating tumors is to place small, radioactive seeds in or near the tumor site. This gives a high radiation dose to the tumor while reducing the radiation exposure in the surrounding healthy tissues. Commonly known implants take the form of loose seeds and spacers that are implanted by needle in the target tissue, or the seeds may be contained within a woven or braided absorbable carrier such as braided suture material and implanted in the target tissue. The loose seeds, however, are dependent on the tissue itself to hold each individual seed in place during treatment, and are known to have a tendency to migrate as the tumor grows or shrinks. Similarly, woven or braided sutures do not assist in the placement of the seeds relative to the target tissue.

Furthermore, loose seeds require time-consuming assembly by the treating physician or a technician prior to implantation into the patient. The loose seeds are typically shipped to the treatment facility directly, where the treatment physician or a technician has to manually load the loose seeds and spacers into a needles prior to the operation. The seeds are placed in a pharmaceutical counting board, and the treating physician has to painstakingly separate and load each seed, followed by the appropriate number of spacers, then additional seeds, etc., into each implant needle. The time-consuming and arduous process needlessly prolongs the physician's exposure to radiation.

Recently, the use of integrated elongate assemblies, or “seed strands,” has grown in popularly. There are many benefits of using seed strands, both pre-implantation and post-implantation. Seed strands are easier to handle and load into needles, as they eliminate the need to manually sort each seed and spacer. The “spacers” in the seed strands are integrated into the strand itself. Unlike loose seeds which can migrate as the tumor grows or shrinks, a seed strand can maintain correct spacing between seeds even after being introduced into the body.

One such seed strand available on the market, Amersham Health Product's Rapid Strand™, is shipped prior to loading into needles. Each Rapid Strand™ is secured in individual grooved strand carriers made of plastic, and the carriers inserted into individual steel shielding cylinders. The cylinders are then individually sealed within a sterilization pouch, which is then sealed in a bag. To use these strands, a treating physician has to cut open each bag and pouch, remove the individual cylinder from within, slide out the grooved carrier, use a scalpel to cut the strand cradled inside the grooved carrier, retrieve the strand, then individually load each strand into a needle. The needles are then inserted in a shielded metal box for sorting and temporary storage prior to the implantation procedure.

The individual packaging of these strands can be cumbersome and can create a lot of potentially bio-hazardous or radioactive packaging to dispose of. The steel cylinders are bulky and heavy to transport. Further, prolonged manipulation by hand is required of the treatment physician to prepare the seed strands for implantation. This exposes him needlessly to the radiation.

What is desired is a delivery system that can hold multiple radioactive seed strands from the sterilization process, through shipping, and until pre-operation preparation, without the need to remove the strands from the carrier. This system would minimize undesirable exposure by the physician to the radiation, and at the same time reduce the amount of packaging needed to safely transport the seed strands in a sterile environment.

It is to be understood that the seed strands must be sterilized vigorously before they can be delivered to the treatment facility. Seed strands are typically sterilized with the use of Ethylene Oxide gas. After sterilization, the strands can be shipped pre-loaded into needles (in which case the needle and the strand were sterilized together), or separate from the needles.

### SUMMARY OF THE INVENTION

It is, therefore, an object of the invention to provide a carrier that can be sterilized by a variety of methods, including the use of Ethylene Oxide (EtO) gas.

It is another object of the invention to provide a transportation system that is small, lightweight and easy to handle, yet has sufficient shielding for transporting a multitude of radioactive seed strands.

It is a related object to provide a transportation system that produces a smaller amount of waste to discard compared to previously known systems.

It is a further object of the invention to provide a system that allows for the shipping of seed strands without the strands being pre-loaded into needles. This allows the treating physician freedom of choice in choosing which make or style of implantation needle to use.

It is a object of the invention to provide a novel delivery system with an integrated measuring and cutting board. This allows the treating physician or a technician to easily and accurately cut strands into pieces of desired length in the operating room, and load the strand pieces into needles immediately prior to implantation, whether a pre-operative dosimeter plan was formulated or not. A related object is to have the capability to ship extra spacer material for the user to configure special loads.

Another object of the invention is to provide a transportation system that keeps the multiple radioactive strands shielded from the user, except for the one strand that the user is currently working on.

It is a further object to provide a carrier that could securely hold a multitude of seed strands.

A related object is to provide a system that can transport one patient's entire requirement of radioactive seed strands in one discrete package.

The above and related objects are addressed by embodiments of the instant invention. Accordingly, an integrated sterilization and transportation system for radioactive seed implants is provided for the shipping of multiple strands without needles. The system is comprised of an outside lead shield envelope, a “fluted” plastic carrier for the individual strands, and an integrated pull-out measuring and cutting board. In the following discussion, it is to be understood that the invention is applicable for both custom-made and generic seed strands, and are not limited to the embodiments disclosed in the inventors' prior patents or any pending applications incorporated herein by reference.

### BRIEF DESCRIPTION OF THE DRAWINGS

FIG. 1 shows in perspective view an integrated sterilization and transportation system for radioactive seed strands according to an embodiment of the invention.

FIGS. 2a-2c depict in sequence perspective views the measuring and cutting board, and plastic carrier being inserted inside the shielded envelope of the system of the invention of FIG. 1.

FIGS. 3a-3d depict in sequence perspective views the pull-out tab being rolled up and folded inside the shielded envelope of FIG. 1.

FIG. 4 shows a perspective view of the sealed system of FIG. 1 inside a sterilized pouch (also shown in FIG. 3d) and being loaded into a container box.

FIGS. 5a-5c show in sequence perspective views the shielded envelope of FIG. 1 being opened, the pull-out tab being unfurled and pulled on to extend the measuring and cutting board, and plastic carrier.

FIGS. 6a-6b show in cross-sectional view the layered construction of the movable assembly of the system in FIG. 1, prior to insertion inside the shielded envelope.

FIGS. 7a-7g show in side cross-sectional view the system of FIG. 1 in operation from assembly, to sterilization, transportation to the end user, and finally the end user retrieving a seed strand.

FIGS. 8a-8c show in side cross-sectional view another embodiment of the movable assembly of the system in FIG. 1, using a flexible tethered hinge.

FIG. 9 shows a perspective view of a seed strand being retrieved from the system of FIG. 1 using surgical tweezers.

FIG. 10 shows a perspective view of a seed strand from the system of FIG. 1 being cut to the desired length on the measuring and cutting board.

FIG. 11 shows a perspective view of a seed strand segment being removed from the system of FIG. 1.

### DETAILED DESCRIPTION OF THE INVENTION

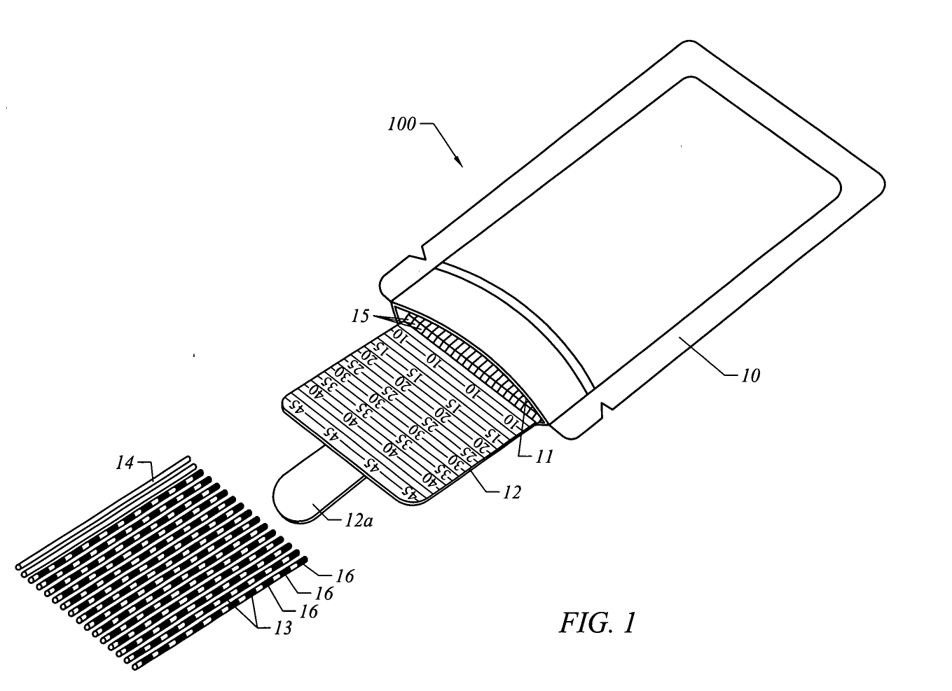

In accordance with an embodiment of the invention, the sterilization and transportation system 100 shown in FIG. 1 is comprised of an outside lead shielded envelope 10, a “fluted,” or pan flute-shaped plastic carrier 11 for the individual strands, and an integrated pull-out measuring and cutting board 12 with attached pull-out tab 12a. The system 100 will securely carry the seed strands through the sterilization process, transportation to the treatment facility, and finally through the preparation of the strands for implantation. Seed strands can be manufactured according to the desired specifications (e.g., number of seeds, seed spacing, type of seed used, etc.) of a patient's pre-operative dosimeter plan. Such techniques are disclosed in the previously discussed patent documents that are incorporated herein by reference. After manufacture, the seed strands are ready to be loaded into the system 100 to be sterilized. In FIG. 1, a multitude of seed strands 13 are shown, along with extra strands of spacer material 14. The strands, typically 10 cm in length, are loaded into the “fluted” plastic carrier 11, so named because of the similarity of the structure to a pan flute, with a bank of parallel tubes, with each tube 15 connected to an adjacent tube. Each tube 15 in the plastic carrier 11 should be of sufficient width to slidably receive a seed strand 13 or spacer strand 14.

Iodine-125 seeds 16 are commonly used for seed strands. The half value thickness of lead for Iodine-125 is 0.025 mm. Thus, a 0.25 mm lead sheet will provide greater than 99% reduction in exposure. In the preferred embodiments of the invention, the shielded envelope 10 of the system 100 should have a lead lining on the inside that is at least 0.25 mm thick, and up to 0.40 mm thick. This would provide shielding that is above required safety standards, while still keeping the shielded envelope 10 relatively lightweight.

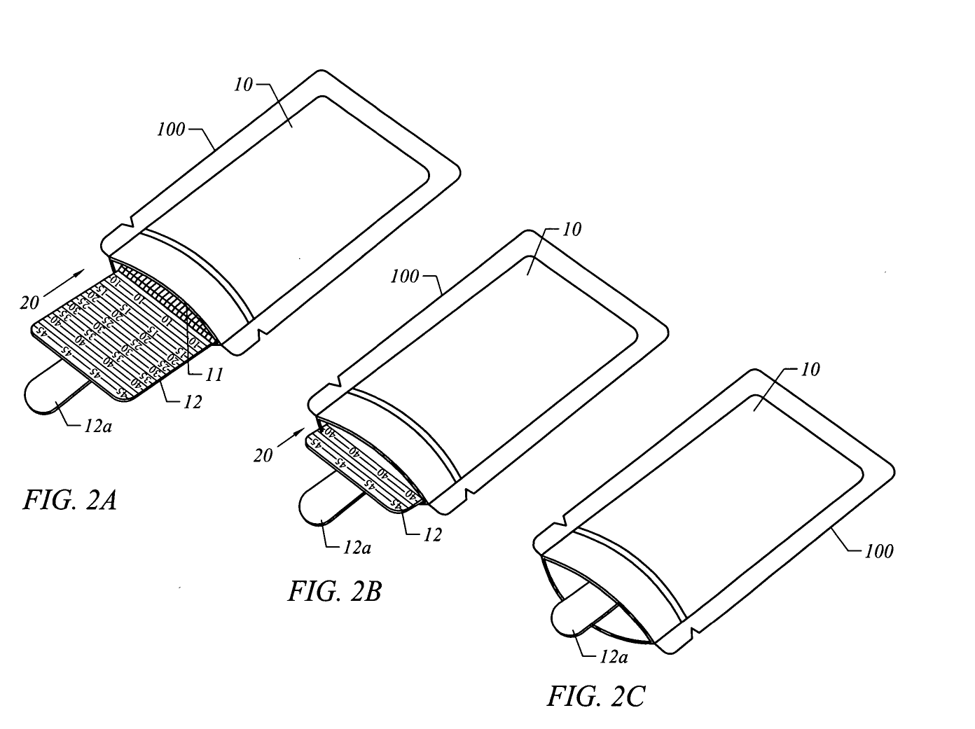

After the strands are loaded into the plastic carrier 11, the measuring and cutting board 12 and plastic carrier 11 are slid inside the lead shielded envelope 10. FIGS. 2a, 2b, 2c show the board 12 and plastic carrier 11 being slid inside the shielded envelope 10 in the direction of arrow 20, until the entire assembly reaches the end of the envelope 10 and only the pull-out tab 12a remains outside (as shown in FIG. 2(c)).

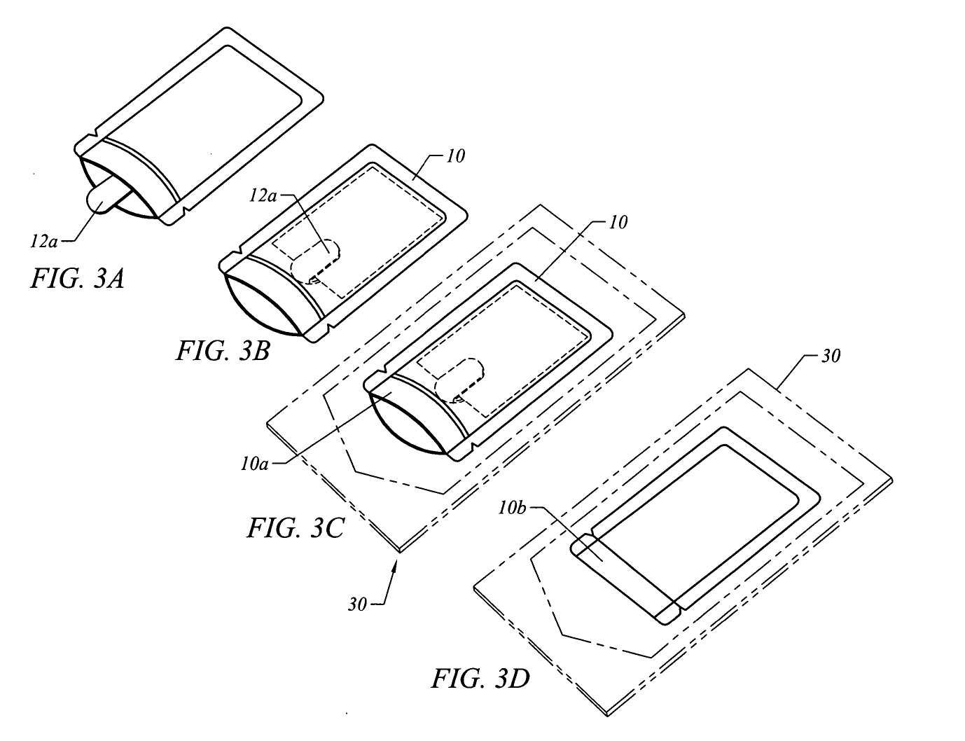

FIG. 3a shows the same configuration as FIG. 2c, with the pull-out tab 12a remaining outside the envelope. Next, the tab 12a is folded back inside the envelope 10 (see FIG. 3b). The tab 12a is preferably made of a material that is sturdy and have a tendency to spring back into the extended form shown in FIG. 3a. The bent back tab (shown in FIGS. 3b and 3c) exerts pressure upwards to hold the lead shielded envelope open during sterilization. The entire open envelope is then inserted inside a pouch 30 for sterilization.

In the preferred embodiment, the pouch 30 is made of a paper type product such as Dupont® Tyvek®, which is chosen for its bacteria growth resistance, tear strength, and puncture resistance, as well as compatibility with existing and emerging sterilization methods. The pouch 30 is sealed and the pouch is loaded into a sterilization chamber for sterilization. The pouch 30 will remain sealed from this point on, until the end-user opens it. In the preferred embodiment, EtO gas sterilization is used, but other methods may also be possible for use with the system 100 as long as a vigorous sterility verification system is in place.

The lead shielded envelope 10 remains open inside the sealed paper pouch 30 during the sterilization process, so that the sterilizing agent can access the system 100, including the plastic carrier 11 and the measuring board 12, the seed strands 13, strands of spacer material 14 and the radioactive seeds 16. After the system 100 and its contents have been thoroughly sterilized, the pouch 30 is removed from the sterilization chamber. The mouth 10a of the shielded envelope 10 is lined with an adhesive, and pressing down on the mouth 10a through the sealed paper pouch 30 closes the shielded envelope 10 by forming a seal 10b. The seed strands 13 are now secured inside the system 100, and the system 100 ready to be loaded into a container for shipping.

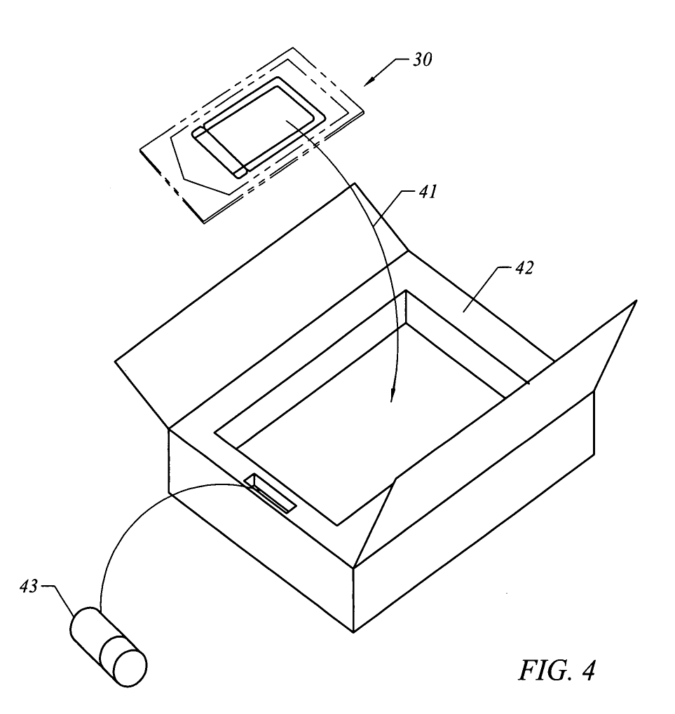

Referring to FIG. 4, the sealed system 100 inside the sterilized paper pouch 30 (also shown in FIG. 3d) is loaded into a container box 41 lined with foam inserts 42. A number of pouches 30 may be shipped in the same container, depending on the size of the order. The container box 41 can additionally carry a container 43 that contains any loose radioactive seeds left over from the manufacturing of the seed strands 13. The container box 41 is sealed and then shipped to the treatment center.

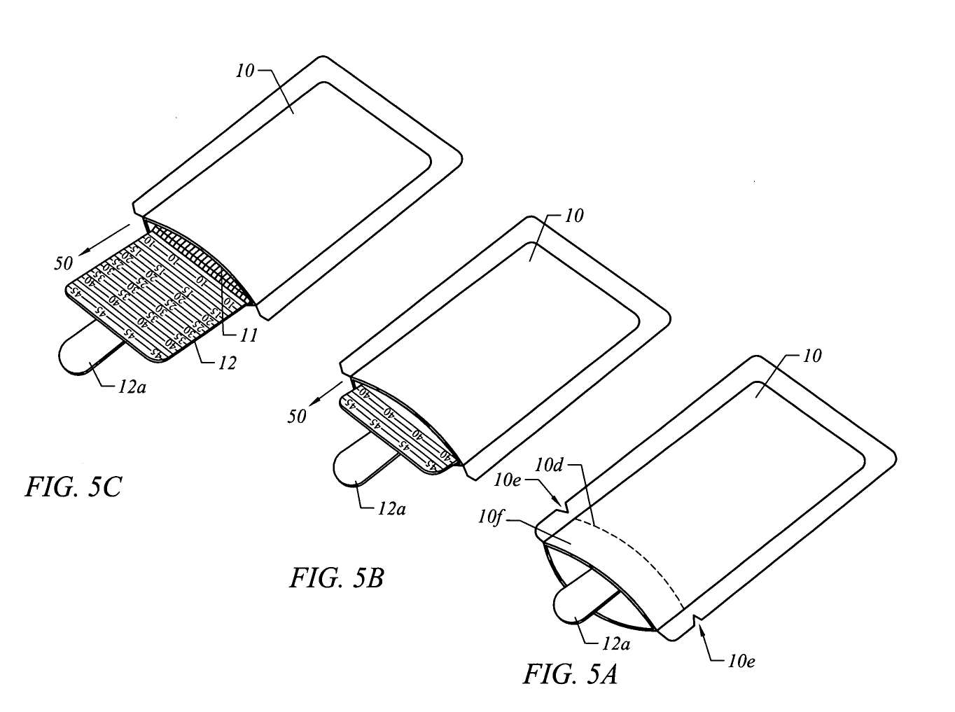

At the treatment center the pouch 30 is removed from the box and brought into a sterile preparation area for preparing and customizing the seed strands 13 for implantation. The lead shielded envelope 10 remains sealed at this point. The pouch 30 is opened and the system 100 removed. Refer to the sequence depicted in FIGS. 5a-5c, the shielded envelope 10 is opened by tearing away the mouth 10a at a perforation 10d marked by notches 10e. The tom-away portion 10f is discarded. The pull-out tab 12a is unfurled until it is sticking out of the now-open shielded envelope. Pulling on the tab 12a in the direction of arrow 50 draws out the measuring and cutting board 12 (see FIG. 5b), which in turn draws out the fluted plastic carrier 11 (see FIG. 5c). Even with the measuring board fully extended (FIG. 5c), the fluted carrier and the radioactive strands 13 located in the carrier 11 remain mostly shielded inside the lead envelope and only the ends of the seed strands are accessible. This design minimizes the radiation exposure to the user during the preparation process.

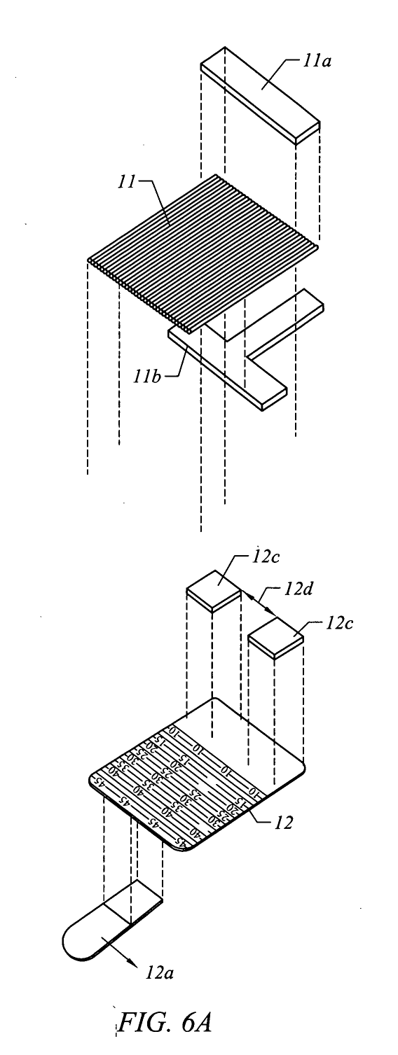

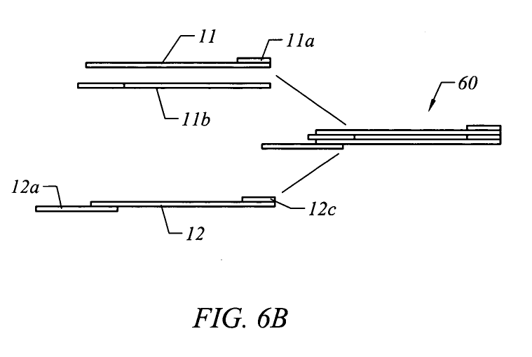

FIGS. 6a to 6b show in detail the layered construction of the system 100. As shown in FIG. 6a, the pull-out tab 12a is attached to the bottom of the measuring and cutting board 12. Towards the rear of the measuring and cutting board 12, away from the pull-out tab 12a, are two flat stops 12c integral with the top surface of the cutting board 12. The two stops form a groove 12d between them. Overlaid on top of the cutting board 12 is the fluted plastic carrier 11. On the bottom side of the plastic carrier 11 is a “T”-shaped plank 11b that runs along the middle of the carrier 11, parallel to the strand carrying tubes 15. The vertical leg of the “T”-shaped plank 11b slidably fits into the groove 12d formed by stops 12c on the cutting board beneath it. On the top side of the plastic carrier 11 is another stop 11a. The cutting board and plastic carrier are laid on top of each other to form an assembly 60, with the vertical leg of the “T”-shaped plank 11b mating with the groove 12d such the board and carrier are in slidable relation to each other.

In operation, pulling on tab 12a pulls the cutting board 12 out of the envelope 10 until the stops 12c come into contact with the “T”-shaped plank 11b. At that point further pulling on the tab 12a cause the fluted plastic carrier 11 and the seed strands 13 to be pulled out of the shielded envelope 10.

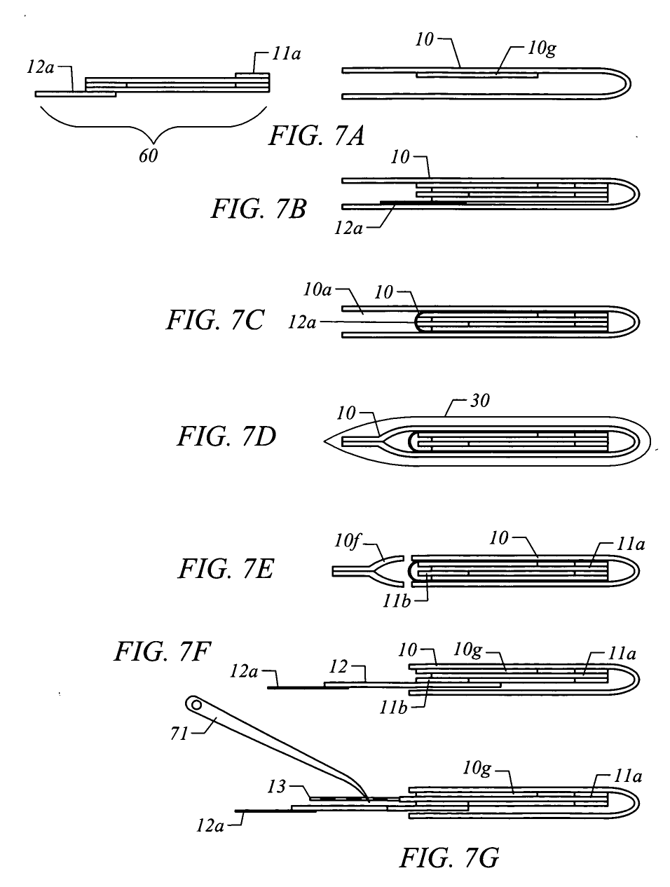

Refer now to FIGS. 7a to 7g, a plastic stop plate 10g integral with the inside of shielded envelope 10 is shown. The assembly 60 is inserted inside the lead shielded envelope 10 until the end of the envelope 10 is reached. FIG. 7b show a completed system 100 (see also FIG. 1) that is ready to receive seed strands. Once the seed strands are loaded, the pull-out tab 12a is bent back inside the shielded envelope 10, as shown in FIG. 7c. (see also FIG. 3b.) The system 100 is then placed inside the paper pouch 30 for sterilization (see FIG. 3c). After sterilization, the mouth 10a of the shielded envelope 10 is sealed by pressing down on the adhesive. The paper pouch 30 remains sealed until it is ready to be used by the user. The system 100 is packed in a box and shipped to the user. When the user is ready to prepare the strands for implantation, the paper pouch 30 is removed, and the front of the shielded envelope 10f torn away and discarded (FIG. 7e). The pull-out tab is pulled out to slidably extend the measuring and cutting board 12. The measuring and cutting board 12 extends out of the envelope 10 until stops 12c reach the horizontal legs of the “T”-shaped plank 11b, as shown in FIG. 7f. The user continues to pull on the tab, and the stops 12c in turn exert a pull on the horizontal legs of the “T”-shaped plank, extending the attached plastic carrier 11. The plastic carrier 11 extends a short distance out of the shielded envelope 10, until the stop 11a on the top side of the plastic carrier 11 is stopped by the plastic stop plate 10g, at which point the assembly 60 is fully extended (FIG. 7g). Note that even in the fully extended state, only the mouth of the fluted plastic carrier 11 extends out of the shielded envelope 10. The seed strands 13 carried within the carrier 11 remains shielded until surgical tweezers 71 are used to retrieve each individual seed strand 13 from the plastic carrier.

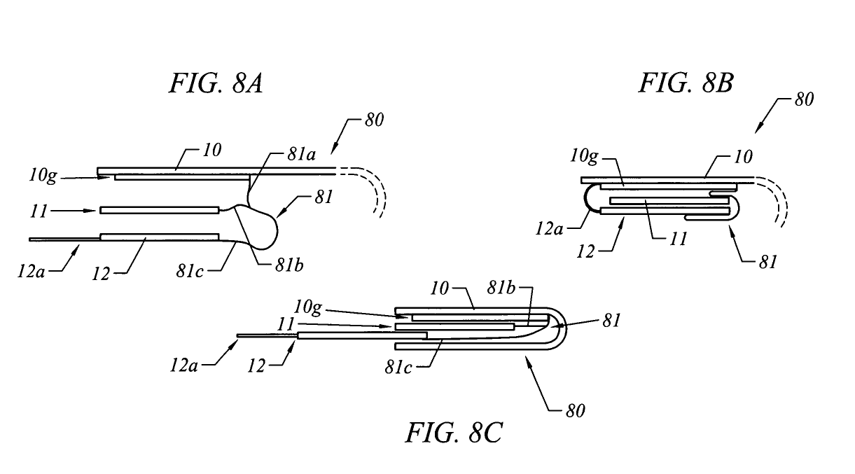

FIGS. 8a to 8c show in cross-sectional view a different embodiment of the assembly of system 100, which is operated in the same way as the embodiment described immediately above. This assembly 80, as shown in FIG. 8a, connects the measuring and cutting board 12, the plastic carrier 11 and the stop plate 10g via a flexible tethered hinge 81. There is no need for the stop means or the “T”-shaped plank in this arrangement. The flexible tethered hinge 81 is a flat piece of material that is comprised of three layers of sheets, 81a, 81b and 81c. Sheet 81a is connected to the stop plate 10g integral with the shielded envelope 10. Sheet 81b is connected to the end of the fluted plastic carrier 11, and sheet 81c connected to the end of the measuring and cutting board 12. The flexible material can be chosen from a variety of materials, including nylon, stiff cardboard, or Dupont® Kapton®. In the assembly's loaded configuration shown in FIG. 8b, the fluted plastic carrier 11 and measuring and cutting board 12 are inserted inside the shielded envelope 10 until the end of the envelope 10 (shown in dotted lines) is reached. The flexible tethered hinge 81 is folded inside the shielded envelope, and the pull-out tab 12a bent back. In FIG. 8c, the measuring board is extended outwards from the shielded envelope by pulling on the pull-out tab 12a, which extends the measuring and cutting board 12 and sheet 81c. Sheet 81c in turn pulls taut the flexible tethered hinge 81 and extends the plastic carrier 11 until the plastic carrier is held taut by sheets 81b and 81a. Like the embodiment described above, the plastic carrier 11 and the seed strands 13 carried within only extends slightly out of the shielded envelope 10.

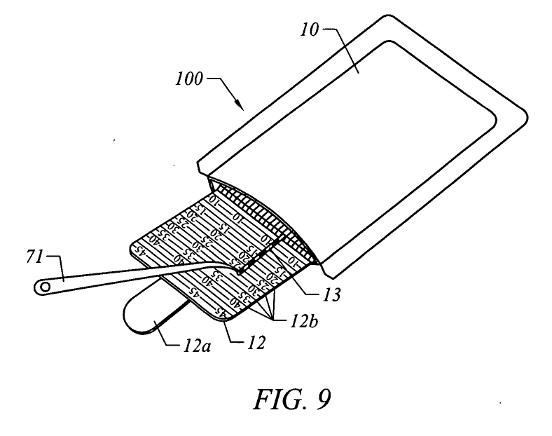

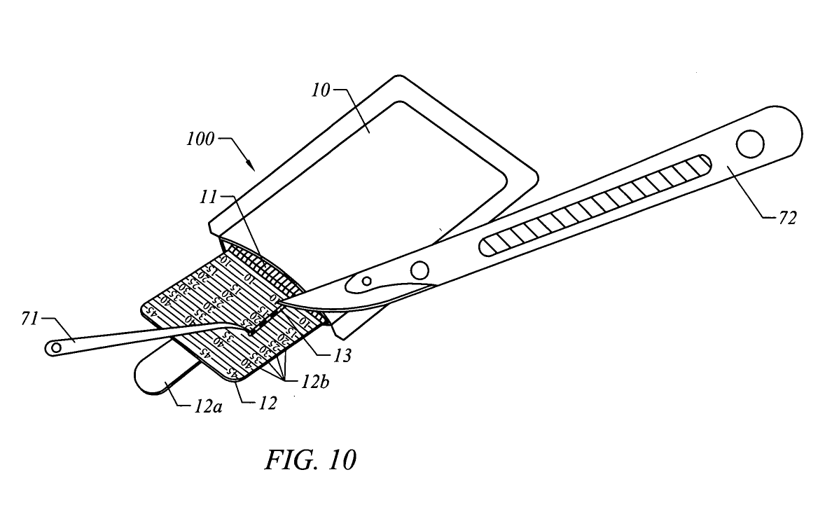

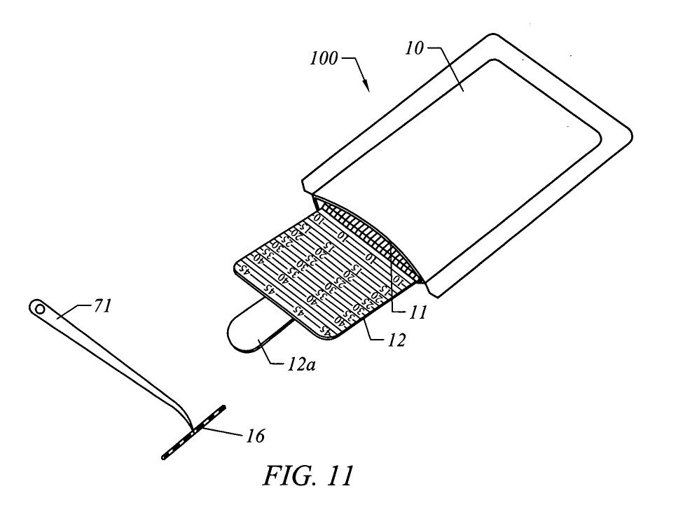

FIG. 9 shows a perspective view of surgical tweezers 71 being used to retrieve one strand 13 from the fluted carrier 11. Using the tweezers 71, the user slides out a seed strand 13 to the desired length as indicated on the measuring and cutting board 12. The lengths are clearly marked on the board 12, preferably in increments of 5 mm. In the preferred embodiment of the invention, groves 12d are etched on the board in order to guide cutting of the strand. In FIG. 10, a scalpel 72 is used to cut the seed strand to the desired length with a groove 12d on the cutting board serving as a guide. Once cut, the seed strand segment is removed from the system 100 (see FIG. 11) and dropped into an implantation needle. Similarly, spacer material in the shape of strands 14 (see FIG. 1) can be retrieved from the fluted carrier 11 and cut to the desired length as needed. Once the seed strand segments and any spacers are assembled in a needle, the seed strands should be implanted within a short period of time in order to maintain their sterility. Unused seed strands can be slid back into the plastic carrier and shielded envelope, and the entire system 100 disposed using disposal means for radioactive material. It is not recommended that unused seed strands be re-sterilized or re-used.

It is to be understood that seed strands manufactured using a variety of techniques can be used with the embodiments of the present invention. In addition to these seed strands referenced to and described above, the following seed strands are appropriate for use with the embodiments of the present invention.

It is desirable for Brachytherarpy seed strands to be flexible and pliable enough to move with the treatment tissue as the tissue shrinks back to pre-operative size. A seed strand also should have sufficiently axial rigidity to allow easy expulsion of the strand from the implantation needle, whereas loose seeds have been known to jam inside an implantation needle.

U.S. patent application Ser. Nos. 10/162,548 and 10/132,930 by the instant inventors disclose two such seed strands, and are incorporated herein by reference.

Patent application Ser. No. 10/132,930, filed Apr. 26, 2002, disclose a seed strand composed of a plurality of tubular shaped, hollow radioactive seeds with a bore. The seed strand is assembled with a polymeric material in the bore between the spaced seeds. In one embodiment, the polymeric material is heated in order to capture the radioactive seed elements at the desired spaced intervals. The strand is axially stiff and radially flexible, and is able to maintain the correct spacing between the seeds as the treated tumor shrinks. The polymeric material is bio-absorbable in living tissue, and is designed to be absorbed by the body in about the same the radioactive seeds become inert.

Patent application Ser. No. 10/162,548, filed Jun. 4, 2002, discloses a seed strand in which a plurality of radioactive seeds can be dispersed in a predetermined array within the strand. In one embodiment, the radioactive seeds are dispersed within pre-fabricated half-shells made of bio-absorbable material, and the half-shells fused to make the seed strand. A system for manufacturing the seed strands is also provided.

Seed strands can be custom-manufactured for each patient from the patient's preoperative dosimeter plan. This allows highly accurate placement of the seeds inside the treatment tissue, in order to provide a precise interstitial radiation dose. Strands are also available in generic, pre-fabricated form. These pre-fabricated implants typically contain seeds spaced at set increments (e.g. 1 cm), and are usually 10 cm long. While generic strands may not offer as precise a placement of seeds for the individual patient, they offer many of the same benefits over loose seeds. Since the seeds are already spaced apart in the strand, all a physician have to do is to cut a strand to the desired length and load it into the implantation needle, without having to manually count and load seeds and spacers. Further, generic strands are more economical than custom-manufactured strands, and using generic stands can also reduce the waiting time for custom-made strands.

It will be appreciated that the instant specification, drawings and claims set forth by way of illustration and not limitation, and that various modification and changes may be made without departing from the spirit and scope of the present invention. Additional aspects, objects and advantages of the invention can be obtained through a review of the appendant claims and figures. It is to be understood that other embodiments can be fabricated and come within the spirit and scope of the claims and the invention.

1. An integrated sterilization and transportation system for radioactive seed strand implants, comprising:
a seed strand carrier adapted to carry a plurality of seed strands and strands of spacer material;
a measuring and cutting board; and
a shielded envelope.

2. The system of claim 1, wherein the strand carrier comprises a plurality of adjacent tubes, each tube being adapted to carry one seed strand or one strand of spacer material.

3. The system of claim 1, wherein said seed strand carrier and measuring and cutting board is slidably housed in said shielded envelope.

4. The system of claim 1, wherein the system is adapted to be sterilized using Ethylene Oxide gas.

5. The system of claim 1, wherein the shielded envelope has a lead lining.

6. The system of claim 5, wherein the lead lining is at least 0.25 mm thick.

7. The system of claim 5, wherein the lead lining is between 0.25 mm and 0.40 mm thick.

8. The system of claim 1, wherein the strand carrier is slidably connected to the measuring board.

9. The system of claim 8, wherein pulling on the measuring board slidably extends the strand carrier.

10. The system of claim 9, further comprising a pull-out tab integral with the measuring and cutting board.

11. The system of claim 9, further comprising means to prevent the strand carrier from advancing beyond the mouth of the shielded envelope.

12. The system of claim 11, wherein said means to prevent the strand carrier from advancing beyond the mouth of the shielded envelope comprises flexible sheet material.

13. The system of claim 11, wherein said means to prevent the strand carrier from advancing beyond the mouth of the shielded envelope comprises plastic stoppers.

14. The system of claim 1, further comprising a pouch, said pouch housing said seed strand carrier, seed strands and strands of spacer material, measuring and cutting board and shielded envelope.

15. The system of claim 14, wherein the pouch is permeable to Ethylene Oxide gas.

16. The system of claim 1, wherein carried seed strands remain shielded by the shielded envelope until the user retrieves each individual strand.

17. The system of claim 1, wherein the measuring and cutting board has markings denoting a desired length of a seed strand segment.

18. The system of claim 17, wherein a desired length of seed strand segment is measured and cut on the measuring and cutting board.

19. The system of claim 17, wherein the measuring and cutting board has marked grooves to aid in the cutting of seed strand segments.

20. The system of claim 1, wherein the system is adapted to carry the entire prescription requirement of one patient.

21. A method of sterilizing and transporting a plurality of radioactive seed strand implants, comprising the steps of:
loading a plurality of seed strands into a strand carrier,
said strand carrier being slidably connected to a measuring and cutting board,
inserting the strand carrier and measuring and cutting board into a shielded envelope,
inserting the shielded envelope into a paper pouch and sealing the paper pouch;
sterilizing the pouch; and
sealing the shielded envelope through the sealed pouch.

22. The method of claim 21, wherein the paper pouch and its contents are sterilized using Ethylene Oxide gas.

23. A method for implanting a plurality of radioactive seed strand implants, comprising the steps of:
receiving a plurality of seed strands in a container assembly;
said container assembly comprising a measuring and cutting board and a seed strand carrier housed inside a shielded envelope,
retrieving a seed strand from the seed strand carrier,
using the measuring and cutting board to measure and cut a desired length of seed strand segment; and
loading said seed segment into an implantation needle.

24. A method of sterilizing a plurality of seed strand implants, comprising the steps of:
loading a plurality of seed strands into a container assembly;
said container assembly comprising a measuring and cutting board, a seed strand carrier adapted to carry a plurality of seed strands, and a shielded envelope to house said measuring and cutting board and seed strand carrier;
sealing the container assembly in a pouch,
sterilizing said pouch; and
sealing said shielded envelope.

25. An integrated sterilization and transportation system for radioactive seed strand implants, comprising:
a seed strand carrier;
at least one seed strand being carried by said strand carrier;
a measuring and cutting board; and
a shielded envelope.

26. The system of claim 25, further comprising at least one strand of spacer material being carrier by said strand carrier.

27. The system of claim 25, further comprising a pouch, said pouch housing said seed strand carrier, at least one seed strand, measuring and cutting board and shielded envelope.

28. The system of claim 25, wherein said strand carrier is slidably connected to said measuring and cutting board.

29. The system of claim 25, wherein said seed strand carrier, at least one seed strand and measuring and cutting board are slidably housed inside said shield envelope.

30. The system of claim 25, wherein the seed strand carrier comprises a plurality of adjacent tubes, each tube being adapted to carry one seed strand or one strand of spacer material.

31. The system of claim 25, wherein the system is adapted to be sterilized using Ethylene Oxide gas.

32. The system of claim 25, wherein the shielded envelope has a lead lining.

33. The system of claim 32, wherein the lead lining is at least 0.25 mm thick.

34. The system of claim 32, wherein the lead lining is between 0.25 mm and 0.40 mm thick.

35. The system of claim 28, wherein pulling on the measuring board slidably extends the strand carrier.

36. The system of claim 35, further comprising a pull-out tab integral with the measuring and cutting board.

37. The system of claim 35, further comprising means to prevent the strand carrier from advancing beyond the mouth of the shielded envelope.

38. The system of claim 37, wherein said means to prevent the strand carrier from advancing beyond the mouth of the shielded envelope comprises a flexible hinge.

39. The system of claim 38, wherein said flexible hinge is connected to the strand carrier, measuring and cutting board and shielded envelope.

40. The system of claim 37, wherein said means to prevent the strand carrier from advancing beyond the mouth of the shielded envelope comprises solid stoppers.

41. The system of claim 40, wherein at least one solid stopper is integral with the shielded envelope.

42. The system of claim 27, wherein the pouch is permeable to Ethylene Oxide gas.

43. The system of claim 25, wherein said at least one seed strand remain shielded by the shielded envelope until the user retrieves each individual strand.

44. The system of claim 25, wherein the measuring and cutting board has markings denoting a desired length of a seed strand segment.

45. The system of claim 44, wherein a desired length of seed strand segment is measured and cut on the measuring and cutting board.

46. The system of claim 44, wherein the measuring and cutting board has marked grooves to aid in the cutting of seed strand segments.

47. The system of claim 25, wherein the system is adapted to carry the entire prescription requirement of one patient.

48. The system of claim 25, wherein the seed strand carrier, the measuring and cutting board and the shielded envelope are slidably connected to each other by a flexible hinge.
